(19) United States
(12) Patent Application Publication
(10) Pub. No.: US 2022/0133217 A1
(43) Pub. Date: May 5, 2022

(54) METHOD AND SYSTEM FOR VISUALIZING A PROPERTY OF CARTILAGE

(71) Applicant: SyntheticMR AB (publ), Linköping (SE)

(72) Inventors: Marcel WARNTJES, Linköping (SE); Per WIDHOLM, Linköping (SE)

(73) Assignee: SyntheticMR AB (publ), Linköping (SE)

(21) Appl. No.: 17/083,971

(22) Filed: Oct. 29, 2020

(51) Int. Cl. A61B 5/00; A61B 5/055; G01R 33/50

(52) U.S. Cl. CPC A61B 5/4514; G01R 33/50; A61B 5/004; A61B 5/055

(57) ABSTRACT

A method of visualizing a property of cartilage, comprising: generating, by a processing circuit, a plurality of slices, based on a magnetic resonance, MR, sequence of a portion comprising cartilage, wherein each slice represents a layer of the portion; for each slice, generating, by the processing circuit, a cartilage line representing cartilage of the slice; for the cartilage line of each slice, generating, by the processing circuit, an image column for visualizing a cartilage property of the cartilage of the slice; and generating, by the processing circuit, a two-dimensional, 2D, image for visualizing the cartilage property of the cartilage of the portion, based on the generated image columns.

### TECHNICAL FIELD

The present document relates to a method and system for visualizing a property of cartilage. Particularly, the present document relates to a method and system for visualizing a property of cartilage, based on magnetic resonance imaging techniques.

### BACKGROUND

Cartilage is a resilient and smooth elastic tissue, a rubber-like padding that covers and protects the ends of bones at the joints, and is a structural component of the rib cage, the ear, the nose, the bronchial tubes, the intervertebral discs, and many other body components. There are different types of cartilage. For example, a hyaline cartilage is a glass-like (hyaline) but translucent cartilage, which is normally pearl-grey in color, and found on joint surfaces. The cartilage is not as hard and rigid as bones, but it is much stiffer and much less flexible than muscles. The cartilage is important to support a smooth motion of the joints. Thus, it is important to assess the properties of the cartilage.

Musculoskeletal (MSK) imaging is commonly used for analyzing bones, joints and associated soft tissues including cartilage, for diagnosing injuries and diseases, based on magnetic resonance imaging (MRI) techniques.

A typical MRI scanner comprises a large, powerful magnet, and can send signals to, e.g., a body portion of a patient. The returning signals can be detected and converted into images of the body portion by a computer device. The images can be obtained in multiple planes (axial, sagittal, coronal, or oblique) without repositioning the patient.

The MRI techniques are generally based on relaxation properties of excited hydrogen nuclei (protons) of an object under test. When the object to be imaged is placed in a powerful, uniform magnetic field of the MRI scanner, the spins of the atomic nuclei of water in the object with non-integer spin numbers within the object all align either parallel or anti-parallel to the magnetic field. From an MR acquisition, several physical properties of the object under test can be determined. And an image can be reconstructed based on an acquired magnetic resonance sequence generated with the excitation.

Proton density (PD) refers to a concentration of protons in a tissue, wherein the protons are the hydrogen nuclei that resonate and give rise to the nuclear magnetic resonance signal. Since most visible tissue protons are resident in water, it is often seen as looking at a water content. The proton density PD of a tissue usually refers to the concentration of protons in the tissue, relative to that in the same volume of water at the same temperature.

The following time constants involved in the relaxation processes, which establish equilibrium following RF excitation, should be introduced in order to understand the MRI techniques. A nuclear magnetic resonance signal is affected by two simultaneous relaxation processes. The loss of coherence of the spin system attenuates the MRI signal with a time constant called a transverse relaxation time (T2). Concurrently, the magnetization vector slowly relaxes towards its equilibrium orientation that is parallel to the magnetic field by a time constant called longitudinal relaxation time (T1). A longitudinal relaxation rate R1 is the reciprocal of the longitudinal relaxation time T1 (R1=1/T1). A transverse relaxation rate R2 is the reciprocal of the transverse relaxation time T2 (R2=1/T2). The relaxation times T1 and T2 are typically measured in milliseconds (ms) or seconds (s). The corresponding relaxation rates R1 and R2 are therefore measured in units of ms⁻¹ or s⁻¹.

Normally, an acquired MRI sequence can result in images of the same anatomical section under different contrasts, such as T1-weighted, T2-weighted and PD-weighted images. The MRI techniques relies on differences in relaxation properties and proton density of the imaged tissue to display the different tissues with contrast, e.g., in different signal intensities or different colors, in the resulting MRI images. The contrast in MR images originates from the fact that different tissues have, in general, different R1 and R2 relaxation rates, and different PD. For example, Warntjes et al. Magn Reson Med. 2008; 60:320-9 teaches that these physical properties, e.g., R1 and R2 relaxation rates and PD, can be acquired by performing a single MR acquisition, to provide quantitative values of the imaged portion.

However, even with the help of the MRI techniques, cartilage assessment can be challenging because the cartilage typically has a complicated three-dimensional (3D) shape. Imaging a joint based on the MRI techniques can result in a stack of slices, each slice representing a layer of the imaged portion, i.e. only a layer of the joint and a layer of the cartilage. Thus, a radiologist has to scroll through a large number of slices, typically 20-30 slices, to analyze the condition of the cartilage of the whole imaged portion, which is not only time consuming, but also ineffective.

Thus, there is a need to provide a method and system to improve the visualization and assessment of cartilage.

### SUMMARY

It is an object of the present disclosure, to provide a new method and system of visualizing a property of cartilage, which eliminates or alleviates at least some of the disadvantages of the prior art.

The invention is defined by the appended independent claims. Embodiments are set forth in the appended dependent claims, and in the following description and drawings.

According to a first aspect, there is provided a method of visualizing a property of cartilage. The method comprises: generating, by a processing circuit, a plurality of slices, based on a magnetic resonance, MR, sequence of a portion comprising cartilage, wherein each slice represents a layer of the portion; for each slice, generating, by the processing circuit, a cartilage line representing cartilage of the slice; for the cartilage line of each slice, generating, by the processing circuit, an image column for visualizing a cartilage property of the cartilage of the slice; and generating, by the processing circuit, a two-dimensional, 2D, image for visualizing the cartilage property of the cartilage of the portion, based on the generated image columns.

Since the cartilage normally has a complicated 3D shape, for a radiologist to evaluate its condition, e.g., its thickness, it is necessary to review and analyze a set of slice images representing different parts, e.g., different layers and different areas of the cartilage, which is very time consuming. Further, it is very ineffective, as the radiologist has to switch between the different slice images for understanding the condition of the whole volume of the cartilage. That is, there is no such straight-forward view for visualizing the cartilage property of the whole imaged portion.

In the present application, a straight-forward view for visualizing the cartilage property of the whole imaged portion can be provided, by visualizing the cartilage property of the 3D portion by the 2D image. Based on the resulted 2D image, a much faster diagnose can be achieved as only one 2D image instead of a set of images is needed.

Further, it can provide a simplified representation of the cartilage property, e.g., its thickness, which can be used for comparing with other patients or patient groups, and/or following disease development.

During the MR imaging process, an imaged portion may be represented by a plurality of slices, each slice representing one of a set of layers of the imaged portion. Each slice may comprise a matrix of image voxels, each image voxel representing one tissue voxel of the layer corresponding to the slice.

In other words, the imaged portion may be divided into a set of layers, and each layer may be further divided into a matrix of tissue voxels. Each tissue voxel may be separated from all of the other tissue voxels by its properties, e.g., its signal intensity. That is, a slice, i.e., a matrix of image voxels corresponding to a matrix of tissue voxels of a layer, may be used to represent the layer. The set of slices may be used to represent the whole imaged portion.

A voxel is a volume element, used to represent a tiny 3D portion in a 3D volume. Voxels are frequently used in the visualization and analysis of medical 3D images. Here, each voxel represents a corresponding tiny volume of the imaged portion. Thus, each voxel may have a value, e.g., signal intensity, and/or a quantification value, e.g., R1 and R2, representing characteristics of the tissues of the corresponding tiny volume of the imaged portion.

A pixel is an element, used to represent a tiny 2D portion in a 2D image. The 3D imaged portion may be sliced into a stack of slices each having a thickness. A voxel may be considered to correspond to a pixel for a given slice thickness. In other words, a voxel can be considered as a volumetric pixel for the given slice thickness. Thus, a 3D image may be converted into a series of 2D images. Consequently, the 3D voxels may be converted into a series of 2D pixels.

The slice may be a 3D slice. That is, each slice may have a thickness. However, the thickness may be ignored during processing such that the slices may be considered as a 2D slice for the given slice thickness. For example, the value of a pixel of a 2D slice may be the averaged value of all the voxels of a 3D slice projecting to the pixel of the 2D slice.

A 2D image may refer to a 2D digital image comprising a plurality of rows and/or columns of pixels, each pixel having a numeric representation for its pixel property, e.g., intensity, color. An image column and an image row may respectively comprise a column or a plurality of columns of pixels, and a row or a plurality of rows of pixels.

The method may further comprise: prior to the step of generating a cartilage line, segmenting, by the processing circuit, the cartilage for generating a cartilage surface representing the cartilage of the portion; wherein for each slice, the cartilage line is an intersection line of the slice and the cartilage surface.

A surface may be a plane, which is not necessarily flat. A cartilage surface representing the cartilage of the portion may be a curved surface.

The cartilage surface may be a surface representing a highest probability of cartilage within a portion. The cartilage surface can be generated by known methods, e.g., segmentation. See for example Shim et al. Radiology. 2009; 251:548-56. Knee cartilage: efficient and reproducible segmentation on high-spatial-resolution MR images with the semiautomated graph-cut algorithm method.

Based on the MR sequence, a computer algorithm can be used to generate a cartilage surface representing a highest probability of cartilage within the imaged portion.

When the cartilage surface is known, the cartilage line of each slice may be considered as a plane, cutting through the cartilage surface, i.e., the intersection line of the slice and the cartilage surface.

The step of generating a cartilage line may comprise: for each slice, segmenting, by the processing circuit, cartilage for generating the cartilage line.

Alternatively, or in combination, a cartilage line may be a line representing a highest probability of cartilage within a slice. The cartilage line can be generated by the same methods, e.g., segmentation, as for generating the cartilage surface.

The step of generating an image column may comprise: for each cartilage point of the cartilage line, determining, by the processing circuit, a cartilage property value; generating the image column comprising a column of pixels, each pixel corresponding to a cartilage point of the cartilage line in order; assigning a property value for each pixel according to a cartilage property value of its corresponding cartilage point.

A cartilage line may comprise a plurality of cartilage points. Each cartilage point may be considered as a sample of the cartilage line. More cartilage points may typically provide a more accurate representation of the cartilage line. Fewer cartilage points may provide a less accurate representation of the cartilage line, but typically a faster processing speed.

The pixels may be arranged according to the order of the cartilage points of the cartilage line. That is, the first cartilage point of the cartilage line may be represented by the first pixel of the column, the second cartilage point of the cartilage line may be represented by the second pixel of the column, etc.

Each cartilage point may correspond to one or more pixels of the image column. Each image column may comprise more than one column of pixels,

The pixels having different property values may be displayed differently, e.g., in different colors or different intensities.

The property of a pixel may be a color or intensity. For example, one pixel corresponding to one cartilage point which has a thin cartilage thickness may be displayed in a darker color, e.g., in black. Another pixel corresponding to another cartilage point which has a thick cartilage thickness may be displayed in a brighter color, e.g., in white or grey.

The step of generating the 2D image may comprise: arranging the image columns side by side in order, such that center pixels of all the image columns are aligned; wherein a center pixel corresponds to a center of any of: the portion, a slice, a cartilage line, and a cartilage surface representing the cartilage of the portion.

The image columns may be arranged according to the order of the slices. That is, the first slice may be represented by the first image column of the 2D image, the second slice may be represented by the second image column of the 2D image, etc.

The center pixel may represent a cartilage point coinciding with any of: a center of the portion, a center of a slice, a center of a cartilage line, and a center of a cartilage surface representing the cartilage of the portion.

The center pixel may represent a cartilage point being closest to any of: a center of the portion, a center of a slice, a center of a cartilage line, and a center of a cartilage surface representing the cartilage of the portion.

Although certain information, e.g., the 3D form of the cartilage, are lost during the representation of the cartilage property by the 2D image of a plurality of image columns, aligning all the image columns at the center pixels can ensure that the resulted 2D image for visualizing the cartilage property can be correctly positioned at the center row (a row of the center pixels) of the resulted 2D image. Other rows of the 2D image may be displayed with a geometrical distortion of different extents. The geometrical distortion may increase the further a pixel is away from the center pixel. The geometrical distortion is comparable to the geometrical distortion of a 2D map of the earth.

Even though the geometrical distortion exists, since the resulting 2D image is correctly positioned at least at the center row and even at a few rows close to the center row, it can still provide valuable information about the relative location and relative size of a region of interest identified in the resulted 2D image.

The cartilage property may be a thickness of a cartilage.

The thickness may be considered as a local thickness of a cartilage of a tiny volume. The thickness may be defined as a distance wherein cartilage presents on a direction perpendicular to any of a cartilage line, a cartilage surface, and/or a plane of a slice.

The distance may be measured by a number of voxels wherein cartilage presents. The number of voxels may be converted into a stand unit, such as millimeter (mm), based on a size of a tissue voxel represented by one image voxel.

The cartilage property may be a signal intensity of a cartilage.

The term “intensity”, also known as “signal intensity”, in the field of MR may refer to a shade of grey of a tissue or of a voxel representing the tissue in an MRI image. Generally, a high intensity means it would look “white” in the MRI image, an intermediate intensity means it would look “grey” in the MRI image, and a low intensity means it would look “black” in the MRI image.

A pixel intensity and/or brightness may be proportional to the signal intensity of a corresponding voxel of the imaged portion. The pixel may be displayed in a color corresponding to the signal intensity of the corresponding voxel of the imaged portion. Different signal intensities may be used to facilitate analyzing the condition of the cartilage.

The method may comprise acquiring, by an MR scanning device, the MR sequence of the portion.

The step of acquiring the MR sequence and the step of generating the plurality of slices may be one step instead of two. It is common that a MR scanner may perform an acquisition and result in a plurality of slices each representing a layer of the imaged portion.

The MR sequence of the portion comprises quantification information of the portion; wherein the quantification information of the portion comprises at least one of: a longitudinal relaxation rate R1, a transverse relaxation rate R2, a longitudinal relaxation time T1, a transverse relaxation time T2, and a Proton Density, PD.

The cartilage property may comprise at least one of: a longitudinal relaxation rate R1, a transverse relaxation rate R2, a longitudinal relaxation time T1, a transverse relaxation time T2, and a Proton Density, PD.

The method may comprise displaying, by a user interface, the 2D image for visualizing the cartilage property of the cartilage of the portion.

The method may comprise: generating, by the processing circuit, an anatomical image comprising a plurality of anatomical slice images, based on the MR sequence of the portion, or based on a different MR sequence of the portion, wherein each anatomical slice image represents a layer of the portion corresponding to one of the plurality of slices; and displaying, by the user interface, at least one of the plurality of anatomical slice images.

Since both the slice and the anatomical slice image may represent a layer of the portion, there may be a one to one relationship between the anatomical slice images and the slices.

The at least one of the plurality of anatomical slice images may be displayed as seen from at least two different viewpoints.

The MR sequence for generating the anatomical image may comprise 3D information of the portion, e.g., the MR acquisition may be a 3D acquisition. Then, any anatomical slice image may be displayed as seen from at least two different directions or viewpoints. The anatomical slice image may be displayed to render a 3D effect as it is possible to view the slice from various directions.

The method may comprise: when an image column of the 2D image is selected, marking, by the processing circuit, a corresponding anatomy slice image.

The method may comprise: when an anatomy slice image is selected, marking, by the processing circuit, a corresponding image column of the 2D image.

Based on the one to one relationship between the anatomical slice images and the slices, when one of a pair of corresponding anatomical slice image and the slice is selected, the other one is marked. This may assist a user in correlating a cartilage property image to an anatomy image, e.g., to facilitate the diagnose.

The selection may be done by a user, via the user interface. For example, the user interface may comprise a mouse, a keyboard, a touch screen, a joystick, etc.

The “marking” may refer to displaying a marked part of the image differently from the rest of the image, e.g., with a color or a brightness different from its original color or brightness.

Alternatively, or in combination, the “marking” may refer to displaying a marker for marking a marked part of the image. The marker may comprise a graphic, such as a pointing arrow, a rectangular frame, a ring, etc.

At least a part of the 2D image and at least a part of the anatomical image may be displayed simultaneously.

By displaying at least a part of the 2D image and at least a part of the anatomical image simultaneously, a user may easily correlate a cartilage property image to an original anatomy image, such that the diagnosing of the cartilage can be facilitated.

The at least part of the 2D image and at least part of the anatomical image may be displayed simultaneously. For example, when an image column of the 2D image is selected and displayed, a corresponding anatomy slice image is also displayed. When an anatomy slice image is selected and displayed, a corresponding image column of the 2D image is also displayed.

The at least part of the 2D image and at least part of the anatomical image may be displayed on a same display, e.g., side by side. Alternatively, the at least part of the 2D image and at least part of the anatomical image may be displayed on different displays.

The method may comprise generating, by the processing circuit, an indicator for indicating a size and/or a position of a part of the 2D image related to an anatomical image representing at least a part of the portion.

The indicator may have a length for indicating the size of the part of the 2D image. The indicator may have a length for indicating a distance of the part of the 2D image from a center of the portion.

The indicator may indicate a center of the portion for indicating the position of the part of the 2D image.

The method may comprise: dividing the 2D image into a plurality of regions, and calculating a value representing the cartilage property of at least one of the plurality of regions.

In order to assess the cartilage property of the portion, one region of interest of the 2D image may be selected, and calculate a value representing the cartilage property of the region of interest.

It is also possible to divide the 2D image into a plurality of smaller regions, e.g., right and left regions, anterior and posterior regions, and calculate a value representing the cartilage property of one or more of the smaller regions.

The value representing the cartilage property of a region may be calculated based on the cartilage property values of the cartilage points represented by the pixels of this region. The value may be calculated by averaging these cartilage property values. Alternatively, the value may be determined to be a largest, a least, or a median of the cartilage property values of the cartilage points represented by the pixels of this region.

The cartilage property of the region of the 2D image may be further represented by one value, which can further simplify the representation of the cartilage of the portion. That is, the cartilage property of the portion may be visualized by a set of numerical values instead of the 2D image.

Even though the numerical representation of the cartilage property of the portion may be considered simple, it is a powerful tool in many scenarios. For example, the numerical representation of the cartilage property can be used to provide a fast comparison between a patient's left and right joint, e.g., the left and right knee. It can be used to compare between different patient groups, and/or between a patient group and its healthy peers.

The numerical representations of a same patient may change over time and from scan to scan, which can be used to monitor the disease development and recovery process of a patient.

The values calculated for the one or more regions of the 2D image may be displayed and/or output as a table, e.g., for recording and/or reporting.

The cartilage used as examples of this application is the hyaline cartilage normally found on joint surfaces. However, any other types of cartilage may also be visualized by the method and system described herein.

The portion may comprise a joint.

A joint is a connection between bones in a body, which is constructed to allow for different degrees and types of movement. Some examples of joints are knees, elbows, and shoulders.

The portion may comprise a knee.

According to a second aspect, there is provided a system for visualizing a property of cartilage. The system comprises a processing circuit configured to: generate a plurality of slices, based on a magnetic resonance, MR, sequence of a portion comprising a cartilage, wherein each slice represents a layer of the portion; for each slice, generate a cartilage line representing cartilage of the slice; for the cartilage line of each slice, generate an image column for visualizing a cartilage property of the cartilage of the slice; and generate a two-dimensional, 2D, image for visualizing the cartilage property of the cartilage of the portion, based on the generated image columns.

The system may further comprise a user interface configured to display images.

The user interface may be configured to output information, such as texts, sounds, images, etc.

The user interface may be configured to receive input, e.g., a command, from a terminal or an input device via a wire or wirelessly. The user interface may be configured to receive input from a user.

According to a third aspect, there is provided a non-transitory computer readable recording medium having computer readable program code recorded thereon which when executed on a device having processing capability is configured to perform the method.

### BRIEF DESCRIPTION OF THE DRAWINGS

FIGS. 1a-1b are proton density weighted MRI images of a slice.

FIGS. 2a-2b are 2D images for visualizing a cartilage property.

FIG. 3a-3c illustrate measurements of various cartilage properties of the slice of FIGS. 1a-1b.

FIGS. 4a-4c are 2D images for visualizing cartilage properties.

FIGS. 5a-5d are examples of 2D image and anatomical slice images.

FIG. 6 is an example of numerical representation of a 2D image for visualizing a cartilage property.

FIG. 7 is an example of a schematic block diagram of a system for visualizing a cartilage property.

### DESCRIPTION OF EMBODIMENTS

The present invention will now be described more fully hereinafter with reference to the accompanying drawings, in which currently preferred embodiments of the invention are shown.

In connection with FIG. 7, the system 1 for visualizing a property of cartilage will be discussed in more detail. The system 1 comprises a processing circuit 3.

The processing circuit 3 is configured to carry out overall control of functions and operations of the system 1. The processing circuit 3 may include a processor, such as a central processing unit (CPU), microcontroller, or microprocessor. The dynamic MRA system 1 may comprise a memory. The processing circuit 3 may be configured to execute program codes stored in the memory, in order to carry out functions and operations of the system 1.

The memory may be one or more of a buffer, a flash memory, a hard drive, a removable medium, a volatile memory, a non-volatile memory, a random access memory (RAM), or another suitable device. In a typical arrangement, the memory may include a non-volatile memory for long term data storage and a volatile memory that functions as system memory for the system 1. The memory may exchange data with the processing circuit over a data bus. Accompanying control lines and an address bus between the memory and the processing circuit also may be present.

Functions and operations of the system 1 may be embodied in the form of executable logic routines (e.g., lines of code, software programs, etc.) that are stored on a non-transitory computer readable medium (e.g., the memory) of the system 1 and are executed by the processing circuit 3. Furthermore, the functions and operations of the system 1 may be a stand-alone software application or form a part of a software application that carries out additional tasks related to the system 1. The described functions and operations may be considered a method that the corresponding device is configured to carry out. Also, while the described functions and operations may be implemented in software, such functionality may as well be carried out via dedicated hardware or firmware, or some combination of hardware, firmware and/or software.

The system 1 may comprise an MR scanning device 2. The MR scanning device 2 may be configured to acquire an MR sequence of a portion comprising a cartilage. A plurality of slices may be generated based on the MR sequence of the portion, wherein each slice represents a layer of the portion.

The system 1 may comprise a user interface 4. The user interface 4 may be configured to output data and information, e.g., the 2D image for visualizing the cartilage property of the cartilage of the portion, the anatomical image, and/or a value representing the cartilage property of one region of the 2D image. The user interface 4 may be configured to receive data and information, such as a command, from one or several input devices. The input device may be a computer mouse, a keyboard, a track ball, a touch screen, or any other input device. The user interface 4 may send the received data and information to the processing circuit 3 for further processing.

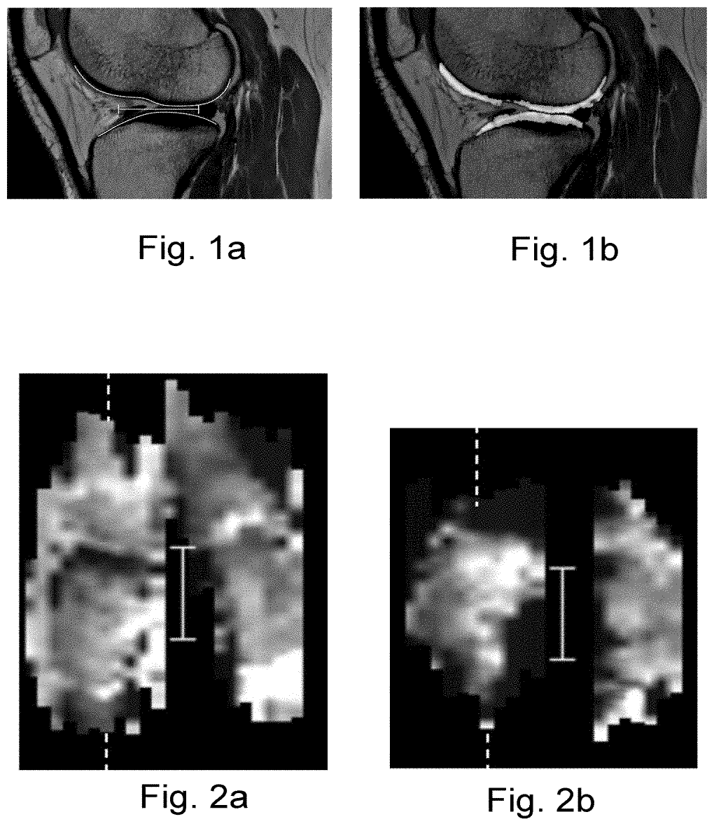

In connection with FIGS. 1a-1b, the cartilage line representing cartilage of a slice will be discussed in more detail.

FIG. 1a is a sagittal PD weighted MRI image of a slice representing a layer of a portion comprising a knee. In FIG. 1a, the upper bone part belongs to a femur, and the lower bone part belongs to a tibia. Two cartilage lines respectively illustrates the cartilage covering the femur and tibia part in this layer of the portion.

The cartilage line may represent a highest probability of cartilage of this layer of the portion. The cartilage line may be generated by segmenting cartilage for this slice, e.g., by an algorithm.

Alternatively, a cartilage surface representing a highest probability of cartilage of the portion may be firstly generated, e.g., by an algorithm. Then, the cartilage line of this slice may be an intersection line of the slice, as a plane, and the cartilage surface. That is, two cartilage surfaces respectively representing the cartilage around the femur and the tibia may be firstly generated. Then, two intersection lines of the slice, as a plane, cutting through these two cartilage surfaces may be considered as the two cartilage lines for this slice.

In FIG. 1a, between the femur and the tibia, there is provided an indicator. This indicator may have a length, as shown in FIG. 1a. The indicator may be used to indicate a size and/or a position of a part of this image. For example, the indicator may indicate a center of this image. The indicator may also indicate a distance of a pixel to a center of this image.

The length of the indicator may be determined to be a fixed value, e.g., 1 cm. The length of the indicator may be determined relatively to the imaged portion. For example, the length of the indicator in FIG. 1a is equivalent to a mean diameter of a menisci (not shown) in a sagittal plane.

A sagittal plane, also known as a longitudinal plane, is an anatomical plane which divides the body or a portion of the body into right and left parts. For example, the sagittal plane may be in the center of the body and split it into two equal halves, or away from the midline and split it into two unequal parts (para-sagittal).

FIG. 1b is a sagittal PD weighted MRI image of the same slice as in FIG. 1a. In FIG. 1b, the two cartilage lines in FIG. 1a respectively representing a highest probability of cartilage around the femur and tibia have been expanded such that all voxels considered to represent cartilage, rather than only the voxels having a highest probability of cartilage, are covered. Consequently, the two cartilage lines of FIG. 1a are expanded to two cartilage areas in FIG. 1b. The brightness of the voxels corresponds to its cartilage probability. The higher the probability, the brighter the voxel appears. In FIG. 1b, a local area in the center part of femur with a thinner cartilage due to traumatic injury is visible.

In connection with FIGS. 2a-2b, the image column and the 2D image for visualizing a cartilage property will be discussed in more detail.

FIGS. 2a-2b are two 2D images for visualizing a cartilage property of the cartilage around the femur and tibia of the portion comprising the layer represented by the images of FIGS. 1a-1b, respectively. The cartilage property visualized in FIGS. 2a-2b is a cartilage thickness.

The cartilage thickness of the upper cartilage line around the femur in FIG. 1a has been converted into an image column of the 2D image of FIG. 2a. The cartilage thickness of the lower cartilage line around the tibia in FIG. 1a has been converted into an image column of the 2D image of FIG. 2b. These two image columns in FIGS. 2a-2b corresponding to the cartilage lines in FIG. 1a are marked by a dashed line.

Each of the cartilage lines of FIG. 1a may comprise a plurality of cartilage points. For each cartilage point, a cartilage property value, e.g., a cartilage thickness, may be determined. This set of cartilage property values may be considered as a representation of the cartilage property of the cartilage line. Consequently, more cartilage points of the cartilage lines may provide a more accurate representation of the cartilage line. Fewer cartilage points of the cartilage lines may provide a less accurate representation of the cartilage line, but typically a faster processing speed.

For example, each cartilage point of the upper cartilage line of FIG. 1a may correspond to at least one pixel of the image column marked by the dashed line in FIG. 2a. The at least one pixel of the image column may have a property, e.g., brightness, and its property value may be determined based on the cartilage property value of its corresponding cartilage point. The pixels having different property values may appear, i.e. may be displayed, differently, e.g., in different colors or different intensities. Thus, the differences of the cartilage property may be obvious only by viewing the 2D image.

In FIGS. 2a-2b, the cartilage thickness of each cartilage point is shown as brightness of the corresponding pixel(s).

In other words, one cartilage line may be sampled to a plurality of cartilage points. Then the cartilage property, e.g., a cartilage thickness, of the plurality of cartilage points may be visualized by the appearance of an image column of the 2D image.

The pixels of an image column of the 2D image may be arranged according to the order of the cartilage points of the cartilage line. That is, the first cartilage point of the cartilage line may be represented by the first pixel of the column, the second cartilage point of the cartilage line may be represented by the second pixel of the column, etc.

The same procedure can be repeated for all the slices (not shown), such that cartilages lines are also generated for other slices and the corresponding image columns for these cartilage lines can be generated in order to generate the 2D image.

The image columns may be arranged side by side in an order according to the slices such that the first slice may be represented by the first image column of the 2D image, the second slice may be represented by the second image column of the 2D image, etc.

A center pixel of each image column can be identified. The center pixels of all the image columns already arranged in order may be aligned to generate the 2D image comprising the image columns. That is, all the image columns are aligned in their center pixels such that the 2D image has a center row comprising the center pixels.

The center pixel may correspond to any of: a center of the portion, a center of a slice, a center of a cartilage line, and a center of a cartilage surface representing the cartilage of the portion. For example, the center pixel may correspond to a cartilage point coinciding with any of the centers, or the center pixel may correspond to a cartilage point being closest to any of the centers.

Thus, the 2D images of FIGS. 2a-2b can visualize the cartilage property of the portion. Although certain information, e.g., the 3D form of the cartilage of the portion, may be lost during the representation of the cartilage property by the 2D image, aligning all the image column together at the center pixels can ensure that the resulted 2D image for visualizing the cartilage property is at least correctly positioned at the center row (a row of the center pixels) of the resulted 2D image. Other rows of the 2D image may be displayed with a geometrical distortion of different extents. The geometrical distortion of the 2D image may increase the further a pixel is away from the center pixel.

Even though the geometrical distortion does exist, since the resulted 2D image is at least correctly positioned at the center row, it can still provide valuable information about the relative position and relative size of a region of interest identified in the resulted 2D image.

The indicator in FIG. 1a is reproduced in FIGS. 2a-2b as a reference. The indicator in FIGS. 2a-2b may have a same length as in FIG. 1a, such that it can provide a reference in terms of distance and size. The indicator in FIGS. 2a-2b may have a same relative position as in FIG. 1a, such that it can provide a reference in terms of position.

For example, in FIG. 2a, an area of darker colors (hypo-intense area) across the dashed line on the left side of the indicator can be identified, which represents cartilage of a thin thickness. This hypo-intense area corresponds to the thinner cartilage area in the center femur in FIG. 1b.

Another example of cartilage property is a signal intensity. The term “intensity”, also known as “signal intensity”, in the field of MR refers to a shade of grey of a tissue or of a voxel representing the tissue in an MRI image. Generally, a high intensity means it would look “white” in the MRI image, an intermediate intensity means it would look “grey” in the MRI image, and a low intensity means it would look “black” in the MRI image.

It is known that the acquired MR sequence has already defined the characteristics of different tissues, including the cartilage, in terms of signal behaviors of the plurality of images, e.g., signal intensities in the images. Thus, the cartilage property can also be the signal intensity of the cartilage of a portion.

For visualizing the cartilage properties, such as cartilage thickness and signal intensity, the MR sequence does not need to comprise any quantification information of the portion.

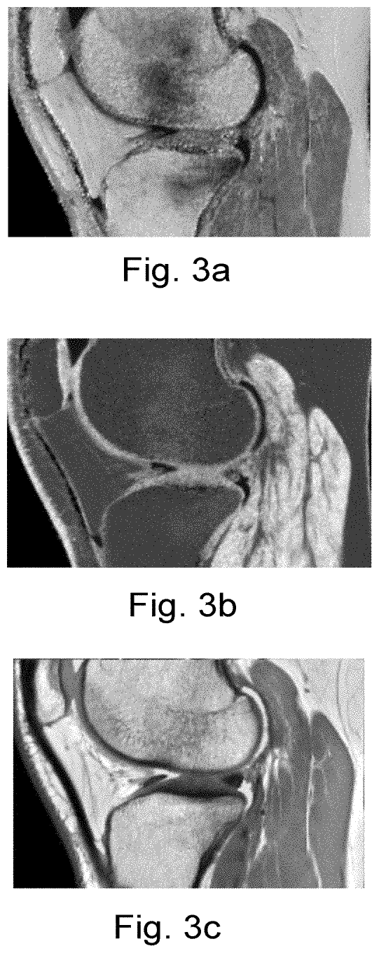

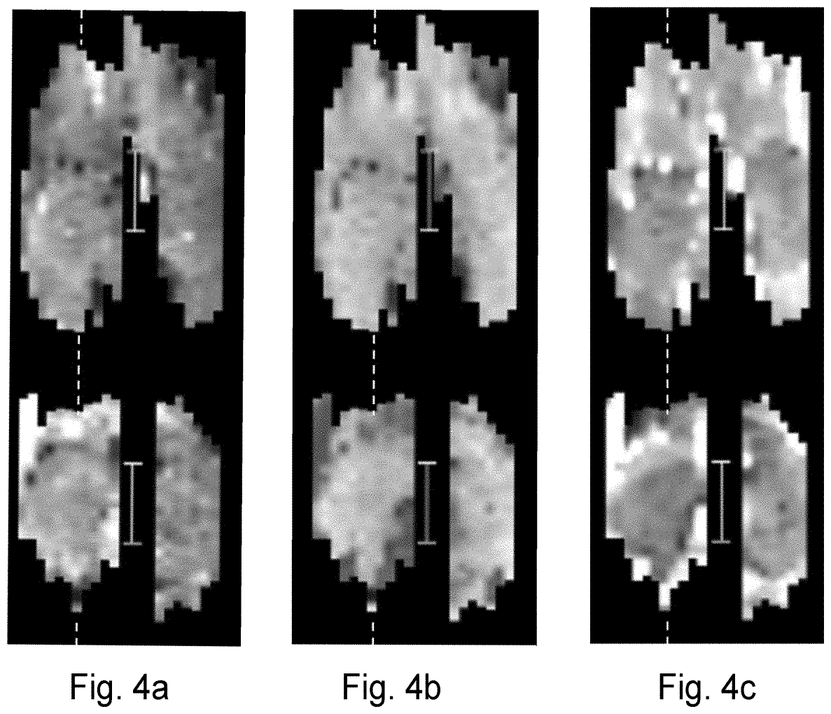

In connection with FIGS. 3a-3c and 4a-4c, examples of different cartilage properties will be discussed in more detail.

FIG. 3a-3c illustrate measurements of different cartilage properties of the slice of FIGS. 1a-1b, based on the MR sequence.

The MR sequence may comprise quantification information of the portion. The quantification information of the portion may comprise at least one of: a longitudinal relaxation rate R1, a transverse relaxation rate R2, a longitudinal relaxation time T1, a transverse relaxation time T2, and a Proton Density, PD. The longitudinal relaxation rate R1 is an inversion of the longitudinal relaxation time T1. The transverse relaxation rate R2 is an inverse of the transverse relaxation time T2.

The cartilage property may be any of: a longitudinal relaxation rate R1, a transverse relaxation rate R2, a longitudinal relaxation time T1, a transverse relaxation time T2, and a Proton Density, PD.

The quantification value for each voxel, including the R1, R2, T1, T2 and PD, may be measured in different known ways, based on the MR sequence comprising quantification information. For example, the R1 and R2 values may be calculated by generating R1 and R2 maps, respectively. When the quantification values comprise additional parameters, e.g., a PD value, additional maps, e.g., a PD map, may be generated. The R1, R2 and PD maps describe the signal behavior of the slices resulted from the MR sequence.

These quantification values of the voxel may be used to determine the tissues of the tiny volume represented by the voxel. That is, it is possible to tell whether a voxel represents cartilage, muscles or bones, based on the quantification values of this voxel. A common way is to generate R1, R2 or PD weighted MRI images.

FIG. 3a is measurement of R1 on a scale 0-2 s⁻¹ on the same slice of FIGS. 1a-1b. FIG. 3b is measurement of R2 on a scale 0-20 s⁻¹ on the same slice. FIG. 3c is measurement of PD on a scale 0-150% on the same slice.

FIGS. 4a-4c are 2D images for visualizing a cartilage property of the cartilage of the same portion. The cartilage property of FIGS. 4a-4c is respectively R1, R2 and PD.

FIG. 4a is the 2D image for visualizing R1 of the cartilage of the portion. FIG. 4b is the 2D image for visualizing R2 of the cartilage of the portion. FIG. 4c is the 2D image for visualizing PD of the cartilage of the portion. The R1, R2 and PD values of each cartilage point may be respectively calculated as a mean value at each point of a cartilage line.

In FIGS. 4a-4c, two 2D images respectively representing two cartilages lines (as shown in FIGS. 2a-2b) are combined as one 2D image, wherein the upper part of the 2D image represents the cartilage around the femur, and the lower part of the 2D image represents the cartilage around the tibia. However, as shown in FIGS. 2a-2b, the upper part and the lower part of the 2D images of FIGS. 4a-4c can be processed and displayed individually.

In FIGS. 4a-4c, the image columns corresponding to the slice of FIGS. 3a-3c are marked by a dashed line, as shown in FIGS. 2a-2b.

The indicator in FIG. 1a is reproduced in FIGS. 4a-4c as a reference. The indicator in FIGS. 4a-4c may have a same length as in FIG. 1a, such that it can provide a reference in terms of distance and size. The indicator in FIGS. 4a-4c may have a same relative position as in FIG. 1a, such that it can provide a reference in terms of position.

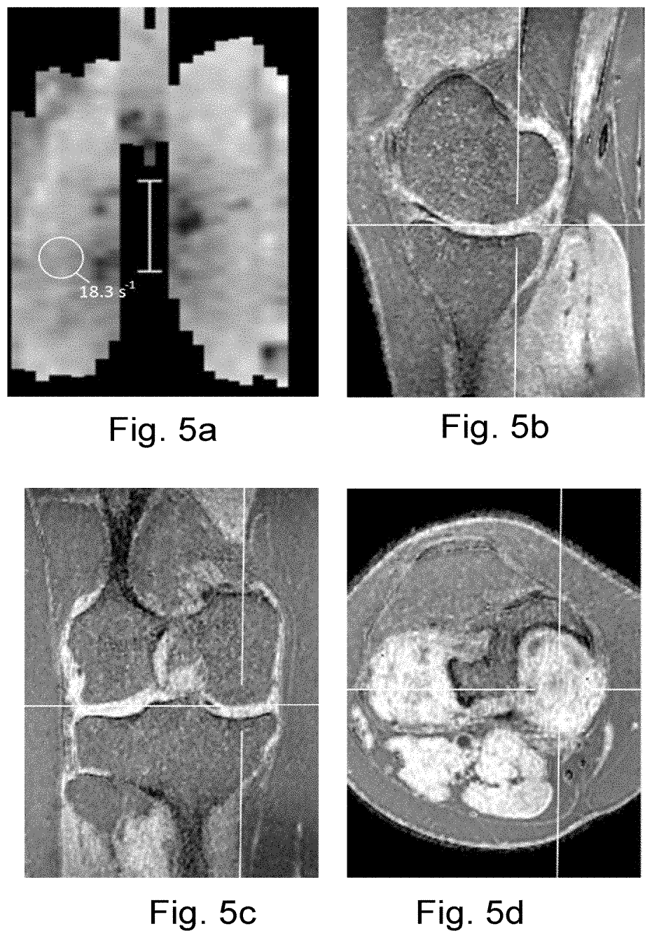

In connection with FIGS. 5a-5b, different examples of 2D image and anatomical images will be discussed in more detail.

FIG. 5a is the upper part of the 2D image for visualizing R2 of the cartilage around the femur, as shown in FIG. 4b.

One region of the 2D image of FIG. 5a is (selected and) marked by a ring as a region of interest (ROI). A value representing a mean value of the cartilage property, i.e. R2, of the ROI, is calculated as 18.3 s⁻¹. The calculated value is depicted close to the ROI in FIG. 5a. The value may be calculated by averaging the R2 values of all the cartilage points corresponding to the pixels within the ROI of the 2D image.

An anatomical image of the portion may be generated based on the MR sequence of the portion, or based on a different MR sequence of the portion. The anatomical image of the portion may comprise a plurality of anatomical slice images, each representing a layer of the portion corresponding to one of the plurality of slices.

The 2D image for visualizing the cartilage property and the anatomical image may be visualized, e.g., side-by-side, on a same display or different displays. This may assist a user in correlating the 2D image visualizing a single cartilage property to the anatomy image.

When one area, e.g., a part of an image column, of the 2D image of FIG. 5a is selected, a corresponding slice is known. For example, the corresponding slice may be a slice corresponding to one column of the ROI.

The corresponding slice may be a slice corresponding to a center column of the ROI. The anatomical slice image corresponding to this corresponding slice can be displayed, correspondingly to the ROI. If there are a few anatomical slice images being displayed simultaneously, this anatomical slice image may be marked such that it appears differently from other displayed anatomical slice images.

The MR sequence of the portion may comprise 3D information of the portion, e.g., the MR acquisition may be a 3D acquisition. Then, any anatomical image, and/or any anatomical slice images may be displayed as seen from different viewpoints/directions, in order to provide different views of the same imaged portion/slice as seen from various directions.

FIGS. 5b-5d are anatomical slice images corresponding to the slice, which corresponds to a center column of the ROI of FIG. 5a. FIGS. 5b-5d visualize the anatomical view of the same slice as seen from different directions. FIGS. 5b-5c are side views and FIG. 5d is a top view. Cross markers are generated in FIGS. 5b-5d for indicating a center of the ROI in different views.

Different ROIs may be selected, e.g., by the user, in the 2D image. The corresponding anatomical slice image may be displayed simultaneously with the 2D image for facilitating diagnosing.

Alternatively or in combination, when an anatomy slice image is selected, an image column of the 2D image corresponding to the selected anatomy slice image is known. The corresponding image column of the 2D image may be displayed. Since normally the displayed 2D image comprising more than one image column, this corresponding image column may be marked such that it appears differently from other image columns.

A selection marker, e.g., a dotted line, may be generated for marking the image column of the displayed 2D image corresponding to the selected anatomical slice image, which is currently displayed. When scrolling through the plurality of anatomical slice images, the selection marker may shift accordingly to indicate the current corresponding image column.

The selection of an anatomical slice image and of an image column of the 2D image, can be done by know way, e.g., by the user clicking on a desired position on a touch screen, or by the user dragging the selection marker to a desired position.

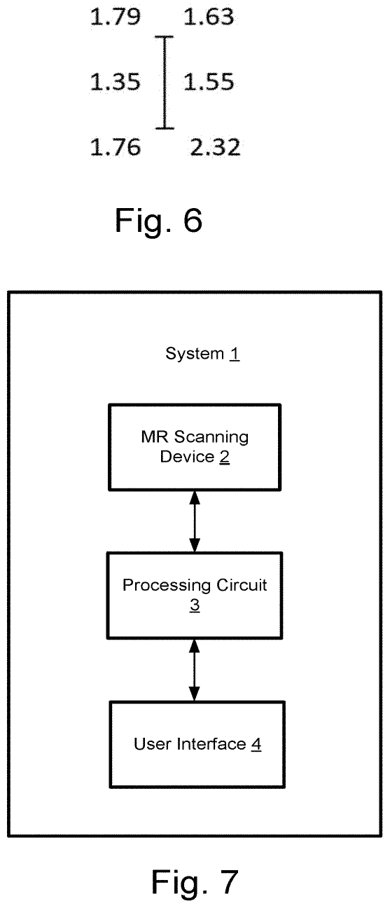

In connection with FIG. 6, an example of numerical representation of a 2D image for visualizing a cartilage property will be discussed in more detail. In FIG. 5a, the ROI is selected and a value representing the cartilage property of the ROI, is calculated. Instead of selecting only one ROI, a plurality of ROIs may be selected. Alternatively, the 2D image may be divided into a plurality of regions, e.g., right and left regions, anterior and posterior regions. A value representing the cartilage property of each region of the 2D image may be calculated.

The value representing the cartilage property of a region of the 2D image may be calculated based on the cartilage property values of the cartilage points corresponding to the pixels of the region. The value may be calculated by averaging the cartilage property values of the cartilage points corresponding to the pixels of the region.

The value may be determined to be a largest, a least or a median of the cartilage property values of the cartilage points corresponding to the pixels of the region.

Alternatively or in combination, the value may be calculated based on the cartilage property values of a selected group of the cartilage points represented by the pixels of the region.

For example, the 2D image visualizing the cartilage thickness around the femur of the portion may be divided into 6 regions around the indicator, e.g., left upper region, left middle region, left lower region, right upper region, right middle region, and right lower region. A mean value of the cartilage property, e.g., cartilage thickness, may be calculated for each region. The six numerical values respectively representing these six regions may visualize the cartilage thickness of cartilage of the portion. The number of regions used here is only an example. That is, the 2D image can be divided into any number of regions.

FIG. 6 is an example of numerical representation of the 2D image of FIG. 2a for visualizing cartilage thickness of the cartilage around the femur. The indicator in FIG. 1a is reproduced in FIG. 6 as a reference. The indicator in FIG. 6 may have a same length as in FIG. 1a, such that it can provide a reference in terms of distance and size. The indicator in FIG. 6 may have a same relative position as in FIG. 1a, such that it can provide a reference in terms of position.

The 2D image visualizing the cartilage thickness of FIG. 2a is divided into 6 regions around the indicator, as described above. For each region, a mean value of the cartilage thickness is calculated in mm, and presented in a corresponding location of the region. For example, the left upper number “1.79” means that the value of the cartilage thickness of the left upper region of the 2D image of FIG. 2a is 1.79 mm.

Thus, the cartilage property of a region of the 2D image can be represented by merely one value, instead of a region of the 2D image, which can further simplify the visualization of the cartilage property. Consequently, a set of values representing the cartilage property of different regions of the 2D image can be used to visualize the cartilage property of the whole imaged portion.

For example, the set of values can be used to compare a patient's left and right joint for a quick analysis. It can also be used to compare between a patient group and its healthy peers. The set of values may change over time and from scan to scan, which can be used to monitor disease development and/or recovery of a patient.

The values of the different regions may be displayed in a table, e.g., for recording and/or reporting.

The imaged portion used in the examples comprises a knee. However, the imaged portion may comprise different joints of a human or an animal, which is constructed to allow for different degrees and types of movement, e.g., an elbow and a shoulder. The imaged portion may also comprise cartilage not attached to closed to any joint.

1. A method of visualizing a property of cartilage, comprising:
generating, by a processing circuit, a plurality of slices, based on a magnetic resonance, MR, sequence of a portion comprising cartilage, wherein each slice represents a layer of the portion;
for each slice, generating, by the processing circuit, a cartilage line representing cartilage of the slice;
for the cartilage line of each slice, generating, by the processing circuit, an image column for visualizing a cartilage property of the cartilage of the slice; and
generating, by the processing circuit, a two-dimensional, 2D, image for visualizing the cartilage property of the cartilage of the portion, based on the generated image columns.

2. The method of claim 1, further comprising:
prior to the step of generating a cartilage line,
segmenting, by the processing circuit, the cartilage for generating a cartilage surface representing the cartilage of the portion;
wherein for each slice, the cartilage line is an intersection line of the slice and the cartilage surface.

3. The method of claim 1, wherein the step of generating a cartilage line comprises:
for each slice, segmenting, by the processing circuit, cartilage for generating the cartilage line.

4. The method of claim 1, wherein the step of generating an image column comprises:
for each cartilage point of the cartilage line, determining, by the processing circuit, a cartilage property value;
generating the image column comprising a column of pixels, each pixel corresponding to a cartilage point of the cartilage line in order;
assigning a property value for each pixel according to a cartilage property value of its corresponding cartilage point.

5. The method of claim 4, wherein the pixels having different property values are displayed differently, e.g., in different colors or different intensities.

6. The method of claim 1, wherein the step of generating the 2D image comprises:
arranging the image columns side by side in order, such that center pixels of all the image columns are aligned;
wherein a center pixel corresponds to a center of any of: the portion, a slice, a cartilage line, and a cartilage surface representing the cartilage of the portion.

7. The method of claim 1, wherein the cartilage property is a thickness of a cartilage.

8. The method of claim 1, wherein the cartilage property is a signal intensity of a cartilage.

9. The method of claim 1, further comprising:
acquiring, by an MR scanning device, the MR sequence of the portion.

10. The method of claim 1, wherein the MR sequence of the portion comprises quantification information of the portion;
wherein the quantification information of the portion comprises at least one of: a longitudinal relaxation rate R1, a transverse relaxation rate R2, a longitudinal relaxation time T1, a transverse relaxation time T2, and a Proton Density, PD.

11. The method of claim 10, wherein the cartilage property comprises at least one of: a longitudinal relaxation rate R1, a transverse relaxation rate R2, a longitudinal relaxation time T1, a transverse relaxation time T2, and a Proton Density, PD.

12. The method of claim 1, further comprising:
displaying, by a user interface, the 2D image for visualizing the cartilage property of the cartilage of the portion.

13. The method of claim 12, further comprising:
generating, by the processing circuit, an anatomical image comprising a plurality of anatomical slice images, based on the MR sequence of the portion, or based on a different MR sequence of the portion, wherein each anatomical slice image represents a layer of the portion corresponding to one of the plurality of slices; and
displaying, by the user interface, at least one of the plurality of anatomical slice images.

14. The method of claim 13, wherein the at least one of the plurality of anatomical slice images is displayed as seen from at least two different viewpoints.

15. The method of claim 13, further comprising:
when an image column of the 2D image is selected, marking, by the processing circuit, a corresponding anatomy slice image;
and/or
when an anatomy slice image is selected, marking, by the processing circuit, a corresponding image column of the 2D image.

16. The method of claim 13, wherein at least a part of the 2D image and at least a part of the anatomical image are displayed simultaneously.

17. The method of claim 1, further comprising:
generating, by the processing circuit, an indicator for indicating a size and/or a position of a part of the 2D image related to an anatomical image representing at least a part of the portion.

18. The method of claim 17,
wherein the indicator has a length for indicating the size of the part of the 2D image, and/or for indicating a distance of the part of the 2D image from a center of the portion;
and/or
wherein the indicator indicates a center of the portion for indicating the position of the part of the 2D image.

19. The method of claim 1, further comprising:
dividing the 2D image into a plurality of regions, and calculating a value representing the cartilage property of at least one of the plurality of regions.

20. The method of claim 1, wherein the portion comprises a joint.

21. A system for visualizing a property of cartilage, comprising a processing circuit configured to:
generate a plurality of slices, based on a magnetic resonance, MR, sequence of a portion comprising a cartilage, wherein each slice represents a layer of the portion;
for each slice, generate a cartilage line representing cartilage of the slice;
for the cartilage line of each slice, generate an image column for visualizing a cartilage property of the cartilage of the slice; and
generate a two-dimensional, 2D, image for visualizing the cartilage property of the cartilage of the portion, based on the generated image columns.

22. The system of claim 21, further comprising:
a user interface configured to display images.

23. A non-transitory computer readable recording medium having computer readable program code recorded thereon which when executed on a device having processing capability is configured to perform the method of claim 1.
